(19) United States
(12) Patent Application Publication
(10) Pub. No.: US 2006/0111259 A1
(43) Pub. Date: May 25, 2006

(54) Detergent composition

(75) Inventors: Debojit Chakrabarty, Bangalore (IN); Rajni Kaushik, Andheri East (IN); Yashodhara Pawar, Andheri East (IN); Nimish Harshadrai Shah, Andheri East (IN)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco Inc, d/b/a UNILEVER

(21) Appl. No.: 11/285,724

(22) Filed: Nov. 21, 2005

(30) Foreign Application Priority Data
Nov. 22, 2004 (IN) 1250/MUM/2004

(51) Int. Cl. A61K 8/02

(52) U.S. Cl. 510/141

(57) ABSTRACT

A detergent composition comprising 10% to 80 weight % of a detergent active, 1% to 60 weight % of a clay belonging to the smectite group, and 0.001% to 10% of zinc pyrithione.

The invention relates to a synergistic detergent composition that provides anti-microbial activity. It particularly relates to a composition for enhanced protection against microbial entities in personal washing.

Conventional detergent bars, based on soap for personal washing typically contain over about 70% by weight total fatty matter (TFM), the remainder being water (about 10% to 20%) and other ingredients such as colour, perfume, preservatives, etc. Structurants and fillers are also typically present in such compositions in small amounts that replace some of the soap in the bar, while retaining the desired hardness of the bar. The commonly used fillers include starch, kaolin and talc. Cleansing compositions are formulated in various forms such as bars, liquids, gels, pastes, etc.

Acne is a condition of the skin, which is highly undesirable especially among the younger population. The cause of acne and its prevention/cure has been widely studied. Internal physiology, the general cleanliness of the skin, the rate of sebum generation, accumulation and removal, the activity of microbial entities e.g bacteria and the weather conditions e.g. the ambient temperature and humidity have all be implicated to varying degrees in the phenomena of acne.

The literature is replete with methods and compositions for eliminating, treating or at least reducing the levels of acne. Various leave on, wash off and face pack type of products have been formulated for the purpose. Use of anti-bacterial agents is one of the preferred routes for alleviation of the condition of acne. Some work has been done on the use of anti-bacterials in wash-off products. Various types of clays have also been suggested both in leave-on type compositions e.g. face-packs for reducing the oiliness of the skin, and also in wash-off compositions.

U.S. Pat. No. 4,118,332 (Colgate-Palmolive, 1978) describes an anti-bacterial detergent composition comprising (1) a diphenyl ether compound and (2) at least one compound selected from the group consisting of 3,4,4′-trichlorocarbanilide and 3,3′,4-trichlorocarbanilides the ratio of (1) to (2) being from 1:3 to 3:1.

U.S. Pat. No. 5,062,994 (Diana Imperatori, 1991) describes a moisture-free skin cleansing composition in tablet form comprising a skin cleansing powder selected from certain anionic or non-ionic surface active agents, and at least one absorbing powder selected from the group consisting of cellulose, modified starches, kaolin, bentonite, talc, silicates, silica, magnesium carbonates, magnesium hydroxides, attapulgite, montmorillonite and mixtures thereof, and a lubricating material and a disaggregating substance.

JP 10-212489 (Kanebo, 1998) describes a cleaning composition comprising one or more anionic surfactant having two chains and two polar groups containing amido groups, and 0.002-5 wt. % microbicide of one or more compounds selected from 2,4, 4′-trichloro-2-hydroxydiphenyl ether, 3,4,4′-trichlorocarbanilide, benzethonium chloride, zinc bis(2-pyridylthio)-N-oxide, piroctoneolamin, photosensitive materials, a C8-22 unsaturated fatty acid such as undecylenic acid and sulfur, and preferably 1-40 wt. % clay mineral.

GB-A-1359492 and EP-A-348015 describe hair treatment compositions containing zinc salts as anti-dandruff agents and clays.

Traditionally, various clays have been topically applied to provide skin benefits. It has now been found by the present inventors that use of a clay belonging to the smectite class of compounds, along with a selective antibacterial agent viz. zinc pyrithione and a detergent active in a washing composition provides synergistic benefit for control of microbial activity. This specific combination surprisingly provides synergistic benefit, while use of other types of clays and anti-bacterials agents do not show this benefit.

It is thus an object of the present invention to be able to provide for a detergent composition that provides synergistic benefit in controlling microbial activity.

It is another object of the present invention to be able to provide a personal wash detergent composition comprising a clay and an anti-bacterial agent which are readily and inexpensively available and therefore enables production of cleansing compositions at low cost.

It is yet another object of the present invention to be able to provide for a personal wash detergent composition which in addition to providing the synergistic anti-bacterial activity also provides for controlling acne or reducing the incidence of acne.

Thus according to the present invention there is provided a detergent composition comprising:
10% to 80 weight % of a detergent active;
1% to 60 weight % of a clay belonging to the smectite group;
0.001% to 10%, preferably 0.01% to 10% of zinc pyrithione;
   and optionally other conventional ingredients.

It is particularly preferred that the clay belonging to the smectite group is a bentonite, and even more preferably sodium bentonite.

The invention is particularly directed to a personal wash detergent composition comprising a detergent active, a clay belonging to the smectite group and zinc pyrithione.

In certain preferred embodiments, the detergent composition is a detergent bar, preferably a soap bar.

The detergent active used in the process may be soap or non-soap surfactants, and in some embodiments is preferably a soap. The term total fatty matter, usually abbreviated to TFM, is used to denote the percentage by weight of fatty acid and triglyceride residues present in soaps without taking into account the accompanying cations.

For a soap having 18 carbon atoms, an accompanying sodium cation will generally amount to about 8% by weight. Other cations may be employed as desired such as for example zinc, potassium, magnesium, alkyl ammonium and aluminium.

The term soap denotes salts of carboxylic fatty acids. The soap may be derived from any of the triglycerides conventionally used in soap manufacture—consequently the carboxylate anions in the soap may contain from 8 to 22 carbon atoms.

The soap may be obtained by saponifying a fat and/or a fatty acid. The fats or oils generally used in soap manufacture may be for example tallow, tallow stearines, palm oil, palm stearines, soya bean oil, fish oil, caster oil, rice bran oil, sunflower oil, coconut oil, babassu oil, palm kernel oil, and others. In the above process the fatty acids are derived from oils/fats selected from coconut, rice bran, groundnut, tallow, palm, palm kernel, cotton seed, soybean, castor etc. The fatty acid soaps can also be synthetically prepared, for example by the oxidation of petroleum or by the hydrogenation of carbon monoxide by the Fischer-Tropsch process. Resin acids, such as those present in tall oil, may be used. Naphthenic acids are also suitable.

Tallow fatty acids can be derived from various animal sources and generally comprise about 1% to 8% myristic acid, about 21% to 32% palmitic acid, about 14% to 31% stearic acid, about 0 to 4% palmitoleic acid, about 36% to 50% oleic acid and about 0 to 5% linoleic acid. A typical distribution is 2.5% myristic acid, 29% palmitic acid, 23% stearic acid, 2% palmitoleic acid, 41.5% oleic acid, and 3% linoleic acid. Other similar mixtures, such as those from palm oil and those derived from various animal tallow and lard are also included.

Coconut oil refers to fatty acid mixtures having an approximate carbon chain length distribution of 8% C8, 7% C10, 48% C12, 17% C14, 8% C16, 2% C18, 7% oleic and 2% linoleic acids (the first six fatty acids listed being saturated). Other sources having similar carbon chain length distributions, such as palm kernel oil and babassu kernel oil, are included within the term coconut oil.

A typical suitable fatty acid blend consisted of 5% to 30% coconut fatty acids and 70% to 95% fatty acids ex hardened rice bran oil. Fatty acids derived from other suitable oils/fats such as groundnut, soybean, tallow, palm, palm kernel, etc. may also be used in other desired proportions.

The composition according to the invention may optionally comprise detergent actives, which may be non-soap detergents and which are generally chosen from anionic, nonionic, cationic, amphoteric or zwitterionic detergent actives. It is preferred that if non-soap detergents are used in the composition of the invention, the non-soap detergent is chosen from an anionic or a nonionic detergent active.

The composition of the invention also comprises a clay belonging to the smectite group of clays. Smectites constitute a group in the class of natural aluminosilicate minerals known as phyllosilicates or layer silicates. Other groups in this class include micas, kaolins, vermiculites, chlorites, talc and pyrophyllite. The phyllosilicate structure consists of layers in which planes of oxygen atoms coordinate to cations such as Si, Al, Mg and Fe to form two-dimensional sheets. The coordination of cations in adjacent sheets typically alternates between tetrahedral and octahedral.

Smectites are characterized by a 2:1 layer structure in which two tetrahedral sheets form on either side of an octahedral sheet through sharing of apical oxygens. As the apical oxygens from the tetrahedral sheet form ditrigonal or hexagonal rings, one oxygen from the octahedral sheet is located on the centre of each ring and is protonated to yield a structural hydroxyl. In 2:1 phyllosilicates, isomorphous substitution of cations having different valencies can lead to charge imbalances within a sheet. These may be partly balanced by the opposite type of charge imbalance in the adjacent sheet (e.g. a positively charged octahedral sheet may offset some of the negative charge associated with a tetrahedral sheet).

The net charge imbalance on a 2:1 layer, if it occurs, is negative. This charge is referred to as the layer charge of the mineral, and is balanced by larger cations (eg., Na⁺, K⁺, Ca⁺² and Mg⁺²) that coordinate to the basal surfaces of the tetrahedral sheets from the adjacent layers. Since these charge-balancing cations are located between adjacent 2:1 layers, they are referred to as “interlayer cations”.

The 2:1 phyllosilicates are distinguished chiefly on the basis of their layer charge.

The compounds/clay materials belonging to the class of smectites is quite large, and especially the Cs exchanged classes of smectites includes hectorite, saponite, and montmorillonite in addition to vermiculite, and several non-exchangeable phyllosilicates.

The preferred clays as per this invention are sodium and calcium montmorillonite, which are part of the smectite group of natural aluminosilicate minerals, and are the most common members. Montmorillonite is the major phase in a type of clays called bentonites. Amongst the calcium and sodium bentonites, sodium bentonite is more preferred.

Clay is preferably present at a concentration of 2% to 40%, more preferably at a concentration of 5% to 30% by weight of the composition.

1-Hydroxy-2-pyridinethione, known as pyrithione, is an aromatic heterocycle related to pyridine via the sulfur and the oxygen of its N-hydroxythioamide group. It forms complexes with most transition metals. It is known for its highly bacteriocidal and fungicidal action. Metallization of the bidentate ligand augments biocidal action, as in the case of complexation of pyrithione with zinc to form zinc pyrithione. Zinc pyrithione is employed as a preservative in various commercial products such as cosmetics or industrial fluids. It is also an effective anti-dandruff agent, and it has been used in several hair care products.

Zinc pyrithione is preferably present in the composition at 0.001% to 10%, preferably 0.01% to 10%, more preferably at 0.05% to 5′ by weight of the composition.

Benefit agents e.g. moisturisers, emollients, sunscreens, or anti ageing compounds may be incorporated in the composition of the invention. Examples of moisturisers and emollients include humectants like polyols, glycerol, cetyl alcohol, carbopol, ethoxylated castor oil, paraffin oils, lanolin and its derivatives. Silicone compounds such as silicone surfactants like DC3225C (Dow Corning) and/or silicone emollients, silicone oil (DC-200 Ex-Dow Corning) may also be included. Sun-screens such as 4-tertiary butyl-4′-methoxy dibenzoylmethane (available under the trade name PARSOL 1789 from Givaudan) and/or 2-ethyl hexyl methoxy cinnamate (available under the trade name PARSOL MCX from Givaudan) or other UV-A and UV-B sun-screens may also be incorporated. Water soluble glycols such as propylene glycol, ethylene glycol, glycerol, may be employed at levels up to 10%.

Other additives such as one or more water insoluble particulate materials e.g. polysaccharides such as starch or modified starches, and cellulose may be incorporated. Minor additives include colour, preservatives and perfumes, may optionally be incorporated.

The composition according to the present invention can be prepared by either the extrusion or cast route. The invention is carried out in any mixer conventionally used in soap/detergent manufacture, and is preferably carried out in a high shear-kneading mixer. The clays according to the invention can be incorporated into the soap or detergent active at any point in the process.

The details of the invention, its objects and advantages are explained hereunder in greater detail in relation to non-limiting exemplary illustrations.

### EXAMPLES

The test suspensions were prepared as shown in Table 1. The test suspensions are representative of a 1% solution/dispersion of a cleaning composition in water.

Measurement of Antibacterial Efficacy

A biofilm based microtitre plate assay was used to determine the antibacterial efficacy of the test suspensions. The efficacy is measured in terms of an index called the growth index [GRI]. The validity of this index was demonstrated by performing the MIC of known anti-bacterials, and its application to biofilm based microtitre plate assay was verified.

Culture Preparation and Growth Conditions.

An axillary isolate of Staphylococcus epidermidis was used for all the studies. The isolate was maintained on sterile CYAgar [Blood agar base 2—42 gm.1⁻¹, dextrose—2 gm.1⁻¹, yeast extract—3 gm.1⁻¹, polysorbate 80—5 gm.1⁻¹]. 24 hrs prior to the study, the isolate was subcultured onto fresh sterile CYAgar and incubated at 37° C. 18 hr old culture of Staphylococcus epidermidis was suspended in sterile phosphate buffered saline [NaCl—8 gm.1⁻¹, KCl—0.2 gm.1⁻¹, Na₂HPO₄—.1.44 gm.1⁻¹, KH₂PO₄—0.24 gm.1⁻¹.] to 0.1 OD₆₀₀ₙₘ for MIC determination and contact kill studies. The culture was suspended in sterile trypiticase soy broth [TSB] with 2% dextrose for in-vitro biofilm assay. The cell density was adjusted to 0.1 OD₆₂₀ₙₘ.

In-Vitro Biofilm Assay

Standardization of biofilm in terms of its attachment to polystyrene wells was initially carried out by crystal violet staining and quantification of cell numbers. Based on this, cells were allowed to form biofilm for 18 hrs in TSB+2% dextrose. After incubating the culture for 18 hrs, the supernatant was discarded using a multichannel pipette. The biofilm was treated with 100 μl of the test suspensions for 1 minute, simulating a wash situation. The test suspension was carefully pipetted out. The biofilm was washed twice with 200 μl sterile phosphate buffered saline, to remove traces of the formulation, while ensuring that the biofilm was not disturbed. Tests were performed in triplicates. After the treatment and washing steps, 200 μl of sterile brain heart infusion broth supplemented with 0.3% glucose and 0.5% Tween was added to each well. A soap control, buffer control, and medium control was also maintained. This assay was performed four times.

A kinetic assay was performed to measure the regrowth of the biofilm in an iEMS reader using the Thermo Labsystems Ascent Software™. Readings were recorded at 37° C. for 24 hrs on an hourly basis at 620 nm.

Calculation

Growth of bacteria is represented by 4 phases—lag phase, exponential phase, stationary phase and death phase. Changes in the surrounding environment such as exposure to antibacterial causes changes in these phases, particularly in the lag and exponential phase. If a culture is grown in the presence of an active, the effect of the antibacterial is directly proportional to the lag time, or the time taken to reach mid log phase and varies inversely to the final increase in cell density. The cumulative effect of the antibacterial over the placebo effect can be determined by the product of the increase in the time taken to reach the mid log phase from the control, and the decrease in the cell density after 24 hrs of growth. Thus the Growth Index is calculated using the formula:
Growth Index=[Time to mid log phase_{control}/Time to mid log phase ₜₑₛₜ]×[(max OD−min OD)test/(max OD−min OD)_{control}]

The data on the GR1 index of the various suspensions was normalised with respect to the control i.e Example 1, and the data is summarised in Table 1.

The lower the normalised GR1 value, the better the antibacterial efficacy.

TABLE 1

Composition (% wt.)

| | Ex 1 | Ex 2 | Ex 3 | Ex 4 |
|---|---|---|---|---|
| Potassium Soap | 1 | 1 | 1 | 1 |
| Bentonite | — | 0.165 | — | 0.165 |
| Zinc pyrithione | — | — | 0.005 | 0.005 |
| Water | To 100 | To 100 | To 100 | To 100 |
| Normalised GRI | 1.0 | 0.34 | 0.36 | 0.11 |

The data presented in Table 1 shows that incorporation of bentonite and zinc pyrithione in the soap composition (Example 4) provides for synergistic benefit in antibacterial efficacy.

A similar set of experiments were conducted where other antibacterials viz. 3,4,4′ trichlorocarbanilide (TCC) and 4,2′,4′-trichloro-2-hydroxy diphenyl ether (TCN) were used instead of zinc pyrithione, and the results are shown in Table 2.

TABLE 2

| | Composition (% wt.) | | | | | |
|---|---|---|---|---|---|---|
| | Ex 1 | Ex 2 | Ex 5 | Ex 6 | Ex 7 | Ex 8 |
| Potassium | 1 | 1 | 1 | 1 | 1 | 1 |
| Soap | | | | | | |
| Bentonite | — | 0.165 | — | 0.165 | — | 0.165 |
| TCC | — | — | 0.003 | 0.003 | — | — |
| TCN | — | | — | — | 0.003 | 0.003 |
| Water | To 100 | To 100 | To 100 | To 100 | To 100 | To 100 |
| Normalised | 1.0 | 0.34 | 0.03 | 0.01 | 0.03 | 0.03 |
| GRI | | | | | | |

The data in Table 2 indicates that antibacterial agents other than zinc pyrithione e.g. TCC or TCN do not provide similar synergistic benefit along with soap and bentonite.

A similar set of experiments were conducted with a clay not belonging to the class of smectites, but belonging to the hormite class of aluminosilicate clays viz., attapulgite. The data generated with attapulgite clay is represented in Table 3.

TABLE 3

Composition (% wt.)

| | Ex 1 | Ex 3 | Ex 9 | Ex 10 |
|---|---|---|---|---|
| Potassium Soap | 1 | 1 | 1 | 1 |
| Attapulgite | — | — | 0.165 | 0.165 |
| Zinc pyrithione | — | 0.005 | — | 0.005 |
| Water | To 100 | To 100 | To 100 | To 100 |
| Normalised GRI | 1.0 | 0.33 | 0.70 | 1.00 |

The data in Table 3 indicates use of a clay other than that in the smectite class does not provide similar synergistic benefit along with soap and zinc pyrithione.

1. A detergent composition comprising:
10% to 80 weight % of a detergent active;
1% to 60 weight % of a clay belonging to the smectite group;
0.001% to 10% of zinc pyrithione.

2. A detergent composition as claimed in claim 1 wherein the clay belonging to the smectite group is selected from calcium or sodium bentonite.

3. A detergent composition as claimed in claim 2 wherein the clay belonging to the smectite group is sodium bentonite.

4. A detergent composition as claimed in claim 2 wherein the clay is a montmorillonite.

5. A detergent composition as claimed in claim 1 wherein the clay is present at 2% to 40% by weight of the composition.

6. A detergent composition as claimed in claim 5 wherein the clay is present at 5% to 30% by weight of the composition.

7. A detergent composition as claimed in claim 1 wherein the detergent active is soap.

8. A detergent composition as claimed in claim 1 wherein zinc pyrithione is present in an amount in the range of 0.01% to 10% by weight of the composition.

9. A detergent composition as claimed in claim 8 wherein zinc pyrithione is present in an amount in the range of 0.05 to 5% by weight of the composition.

10. A detergent composition as claimed in claim 1 wherein the detergent composition is in the form of a detergent bar, preferably a soap bar.
